(19) United States
(12) Patent Application Publication
(10) Pub. No.: US 2026/0092082 A1
(43) Pub. Date: Apr. 2, 2026

(54) ANTIMICROBIAL PEPTIDE HALYMORIN VARIANTS AND METHODS OF USING THE SAME

(71) Applicant: Ke LUO, Holden, MA (US)

(72) Inventor: Ke LUO, Holden, MA (US)

(73) Assignee: Antinbio, Inc., Worcester, MA (US)

(21) Appl. No.: 18/901,435

(22) Filed: Sep. 30, 2024

(51) Int. Cl. C07K 7/08; A23L 3/3526; A61K 8/64; A61K 38/00; A61P 31/04; A61Q 17/00

(52) U.S. Cl. CPC C07K 7/08; A23B 2/762; A61K 8/64; A61P 31/04; A61Q 17/005; A61K 38/00

(57) ABSTRACT

An antimicrobial peptide having an amino acid sequence selected from SEQ ID NOs: 2-12 is provided. The antimicrobial peptide has antimicrobial activity against Gram-negative bacteria, Campylobacter jejuni, Escherichia coli, Salmonella enterica subsp. enterica serovar typhimurium, Salmonella enterica subsp. enterica serovar choleraesuis, Klebsiella pneumonica, and/or Enterobacter cloacae. A composition, pharmaceutical composition, food additive, cosmetic composition, or hygine product having an antimicrobial peptide including an amino acid sequence selected from SEQ ID NOs: 2-12 is also provided. A method of treating an infectious disease caused by bacteria including a step of administering a pharmaceutical composition having the antimicrobial peptide as an active ingredient is also provided.

### SEQUENCE LISTING

The instant application contains a Sequence Listing which has been submitted electronically in Sequence Listing XML format and is hereby incorporated by reference in its entirety. Said Sequence Listing XML copy, created on Sep. 27, 2024, is named S0003-00400 SEQ_3.xml and is 15,604 bytes in size.

### FIELD OF THE INVENTION

The disclosure relates generally to an antimicrobial peptide derived from one or more insects, antimicrobial compositions containing the antimicrobial peptide, and methods of using the same to inhibit microbial infections in a subject and to promote animal growth.

### BACKGROUND

Antimicrobial peptides, also known as host defense peptides, are a class of low-molecular-weight polypeptides that can exhibit broad-spectrum antimicrobial activity against bacteria, fungi, viruses, and protozoa. Antimicrobial peptides are generally induced by external conditions and encoded by certain genes of various biological cells, and play an important role in natural immune defense system. Proteins of insects and fungal origin have been identified as a source of antimicrobial peptides. For example, insects are a natural source of β-hairpin antimicrobial peptides that have a wide spectrum of activity and high resistance to environmental factors such as low pH and high temperature. As another example, plectasin is isolated from the mushroom Pseudoplectania nigrella and belongs to a class of antimicrobial peptides called defensins, which play a role in the body's defense against pathogens. Defensins are considered part of the innate immune response, and act mainly by disrupting the structure of bacterial cell membranes. Defensin-like peptides, such as plectasin, exhibit activity against several methicillin-resistant strains of Staphylococcus aureus.

### SUMMARY

In various embodiments, the present disclosure describes one or more antimicrobial peptides that exhibit exceptional activity against Gram negative bacteria, low to no host toxicity, and high level expression in a recombinant expression system.

In various embodiments, an antimicrobial peptide consisting of an amino acid sequence selected from the group consisting of SEQ ID NOs: 2-12 is provided. In some embodiments, the antimicrobial peptide has antimicrobial activity against Campylobacter jejuni, Escherichia coli, Klebsiella pneumoniae, Salmonella enterica, and/or Enterobacter cloacae, including any subspecies or strains thereof.

In various embodiments, a composition, pharmaceutical composition, food additive, cosmetic composition, or hygine product comprising an antimicrobial peptide halymorin variant consisting of an amino acid sequence selected from the group consisting of SEQ ID NOs: 2-12 is provided. In some embodiments, the antimicrobial peptide is the amino acid sequence of SEQ ID NO: 2. In some embodiments, the antimicrobial peptide is the amino acid sequence of SEQ ID NO: 3. In some embodiments, the antimicrobial peptide is the amino acid sequence of SEQ ID NO: 4. In some embodiments, the antimicrobial peptide is the amino acid sequence of SEQ ID NO: 5. In some embodiments, the antimicrobial peptide is the amino acid sequence of SEQ ID NO: 6. In some embodiments, the antimicrobial peptide is the amino acid sequence of SEQ ID NO: 7. In some embodiments, the antimicrobial peptide is the amino acid sequence of SEQ ID NO: 8. In some embodiments, the antimicrobial peptide is the amino acid sequence of SEQ ID NO: 9. In some embodiments, the antimicrobial peptide is the amino acid sequence of SEQ ID NO: 10. In some embodiments, the antimicrobial peptide is the amino acid sequence of SEQ ID NO: 11. In some embodiments, the antimicrobial peptide is the amino acid sequence of SEQ ID NO: 12. In some embodiments, the antimicrobial peptide halymorin variant is an active ingredient.

In various embodiments, a method of treating an infectious disease caused by bacteria is provided. In some embodiments, the method comprises administering a pharmaceutical composition comprising the antimicrobial peptide halymorin variant as an active ingredient.

Additional features and advantages will be set forth in the detailed description which follows, and in part will be readily apparent to those skilled in the art from that description or recognized by practicing the embodiments as described herein, including the detailed description which follows, the claims, as well as the appended drawings.

### BRIEF DESCRIPTION OF THE FIGURES

The features and advantages of the antimicrobial peptide halymorin variants described herein will be more fully disclosed in, or rendered obvious by the following detailed description of the preferred embodiments, which are to be considered together with the accompanying drawings, wherein:

FIG. 1 is a graph showing a safety profile based on a hemolysis assay for plectasin, halymorin, and exemplary antimicrobial peptide halymorin variants.

### DETAILED DESCRIPTION

In various embodiments, the antimicrobial peptide variants described herein are based on halymorin, which is a peptide obtained from the genomic sequence of the insect halyomorpha picus. Halymorin contains 20 amino acids, has a molecular weight of 2256.6 Da, and a +4.8 positive charge. Halymorin and its variants have a pair of disulfide bonds and a rigid β-hairpin structure, which fold into a β-hairpin Rana box, similar to the structure of other antimicrobial peptides.

In some embodiments, a halymorin variant comprises 10-100 amino acids or amino acid residues. In some embodiments, the halymorin variant comprises 10-30 or 15-25 amino acids or amino acid residues. All ranges include the endpoints and are combinable. In some embodiments, the halymorin variant comprises or consists of 16, 17, 18, 19, 20, 21, or 22 amino acids or amino acid residues. In some embodiments, the halymorin variant comprises or consists of 18 or 20 amino acids or amino acid residues. In some embodiments, the halymorin variant comprises or consists of at least one amino acid sequence selected from the group consisting of SEQ ID NOs: 2-12.

In some embodiments, an amino group (—NH₂) or a methyl group (—CH₃) is added to the C-terminal of the halymorin variant. If the C-terminal of the halymorin variant is amidated, resistance to proteases and positive net charge may be further enhanced. If the C-terminal is methylated, then in vivo stability may be increased based on an improved resistance to exopeptidases, which cleave the peptide from the terminal.

In some embodiments, the N-terminal of the halymorin variant is acetylated or palmitoylated. If the N-terminal is acetylated, superior antimicrobial activity may be achieved and the peptide may be protected from proteolytic degradation. If the N-terminal is palmitoylated, then permeability into cells may be enhanced.

In some embodiments, one or more of the halymorin variants exhibit antimicrobial activity and is commercially viable because the structure consists of short amino acid sequences. In some embodiments, the halymorin variant exhibits strong inner membrane permeability. That is, the halymorin variant exhibits antimicrobial activity by directly permeating into the inner membrane of bacteria. In some embodiments, the halymorin variant lacks cytotoxicity and exhibits minimal or no hemolytic activity.

In some embodiments, the halymorin variant is designed and prepared to have antimicrobial activity against one or more pathogens, including bacteria, such as Gram negative bacteria. In some embodiments, the halymorin variant exhibits antimicrobial activity against one or more species or strains of bacteria, including Campylobacter jejuni, Escherichia coli, Klebsiella pneumoniae, Salmonella enterica, and Enterobacter cloacae, and one or more subspecies or strains thereof.

In some embodiments, the halymorin variant is formulated into an antimicrobial composition. In some embodiments, the composition is a pharmaceutical composition containing a halymorin variant as an active ingredient. In some embodiments, a method for administering the composition to a subject in need thereof is provided. The method of administration is not particularly limited. In some embodiments, the composition is administered intraarterially, intravenously, subcutaneously, intrarectally, intranasally, directly into muscle cells, or via any other parenteral route. In some embodiments, the composition is administered orally (e.g., as a tablet, capsule, pill, suspension, liquid, etc.), nasally, rectally, transdermally, or via injection.

In some embodiments, a dosage of the composition will depend on the activity of the halymorin variant, administration route, severity of the condition to be treated, condition and previous disease history of the subject, etc. For example, the specific administration dosage may be determined based on various factors, including, the age, sex, body type, and body weight of the subject. Further, a process of starting with a lower dosage than is required to achieve the desired therapeutic effect and then gradually increasing the dosage until the desired effect is achieved is within the knowledge of one skilled in the related art.

In some embodiments, the composition may be further processed before being formulated into a pharmaceutically acceptable agent. For example, the composition may be pulverized or ground into particles. In some embodiments, depending on the desired effect, an effective dosage of the halymorin variant may be an amount ranging from about 0.1 to about 10 mg/kg, from about 1 to about 2 mg/kg, from about 0.5 to about 1 mg/kg, etc. In some embodiments, administration may be 1 to 10, 1 to 5, or 1 to 3 times a day. All ranges include the endpoints and are combinable.

In some embodiments, a pharmaceutical composition comprising a halymorin variant is prepared into a formulation of a single dosage form or a multiple dosage form using a pharmaceutically acceptable carrier and/or excipient according to a method typically employed by one of ordinary skill in the art. The formulation may be an oral formulation, such as a powder, granule, tablet, capsule, suspension, emulsion, syrup, aerosol, etc. The formulation may be formulated for external application such as an ointment, cream, etc., or any other pharmaceutical formulation such as a suppository, sterile solution for injection, etc. In some embodiments, the composition may further comprise a dispersant or stabilizer.

In some embodiments, the halymorin variant exhibits antimicrobial activity against one or more microorganisms, including Campylobacter jejuni, Escherichia coli, Klebsiella pneumoniae, Salmonella enterica, Enterobacter cloacae, or one or more subspecies or strains thereof. Accordingly, in some embodiments, the halymorin variant may prevent infection, reduce the chances of infection, or treat diseases caused by bacteria, including but not limited to, respiratory infections, car infections, sinusitis, tonsillitis, urinary tract infections, prostate infections, sexually transmitted infection, gastrointestinal infections, skin infections, food poisoning, candidiasis, typhoid, cholera, etc. Therefore, in some embodiments, the halymorin variant may be used as an active ingredient in a pharmaceutical composition for preventing or treating an infectious disease caused by the microorganisms.

In some embodiments, the halymorin variant is used as an active ingredient in an antimicrobial cosmetic composition. The cosmetic composition may be in the form of a solution, powder, emulsion, lotion, spray, ointment, aerosol, cream, or foam. In some embodiments, the cosmetic composition comprises a carrier that is acceptable in a cosmetic formulation, one or more additional active ingredients, or both. In this context, a “carrier that is acceptable in a cosmetic formulation” refers to an existing compound or composition known to be used in cosmetic formulations; or a compound or composition to be developed, which lacks toxicity, instability, or irritability when applied to the skin of a subject. Examples of an additional active ingredient include steroids, salicylic acid, benzoyl peroxide, retinol, vitamin C, vitamin E, alpha hydroxy acids, dimethicone, and petrolatum.

As used herein, the term “skin” includes the face, the scalp, and the entire body. For example, the cosmetic composition may be prepared as a shampoo, rinse, treatment, hair restorer, etc., for application to the scalp. For application to the face or entire body, the composition may be prepared as a body cleanser, soap, lotion, etc.

In some embodiments, the carrier is included in an amount ranging from about 1 to about 99.99 wt %, or from about 90 to about 99.99 wt %, based on the total weight of the cosmetic composition. For example, in some embodiments, the carrier further comprises an alcohol, oil, surfactant, fatty acid, silicone oil, humectant, moisturizer, viscosity modifier, emulsifier, stabilizer, sunscreen, UV absorbent, colorant, and/or fragrance, etc.

In some embodiments, the cosmetic composition further comprises glycerin, butylene glycol, propylene glycol, polyoxyethylene hydrogenated castor oil, ethanol, triethanolamine, etc. In some embodiments, the composition may contain (for example, a trace amount) of an antiseptic, fragrance, colorant, or purified water.

In some embodiments, the halymorin variant may be used as an active ingredient of a hygiene product. In such embodiments, the hygiene product may be a wet wipe, hand sanitizer, mouthwash, oral antiseptic, toothpaste additive, etc. The halymorin variant should be used in an amount that is effective for inhibiting microbial growth.

In some embodiments, the halymorin variant is used as an active ingredient in a composition for cleaning, disinfecting, or inhibiting microbial growth on a surface. Examples of surfaces, which may be contacted with the halymorin variant, include one or more surfaces of a manufacturing plant or equipment used therein, including, e.g., dairies, chemical processing plants, pharmaceutical processing plants, water sanitation systems, oil processing plants, food processing plants, paper pulp processing plants, water treatment plants, and cooling towers. The halymorin variant should be used in an amount that is effective for cleaning, disinfecting, and/or inhibiting microbial growth on the surface.

In some embodiments, the halymorin variant is used as an active ingredient of food, such as an antimicrobial food. In such embodiments, the halymorin variant can be used in an antimicrobial food or as a feed additive because the halymorin variant exhibits antimicrobial activity against Gram negative bacteria.

In such embodiments, the type of food is not particularly limited. Examples of the food to which the substance can be added include a drink or beverage, including an alcoholic beverage, meat, sausage, bread, biscuit, rice cake, chocolate, candy, snack, pizza, noodles, gum, soup, a diary product, such as yogurt or ice cream, etc. In some embodiments, the food is a vitamin supplement and other health-functional food. The halymorin variant may be added to a food as is or it can be mixed together with other food ingredients. The adequate amount of the active ingredient may be determined depending on the purpose of use (e.g., for prevention or treatment). In some embodiments, for example, the halymorin variant is added in an amount ranging from about 0.01 to about 50 wt. %, or from about 0.1 to about 20 wt. %, or from about 0.1 to about 10 wt. %, or form about 0.1 to about 1 wt. %, wherein the amount is based on the total weight of the food. All ranges include the endpoints and are combinable. In some embodiments, the amount of the active ingredient is smaller than the above-described range. For example, a smaller amount may be used when the composition is used for health or hygiene or otherwise used for a long period of time. In some embodiments, a larger amount of the active ingredient may be used. For example, a larger amount may be used when there are no safety concerns.

In some embodiments, the halymorin variant is used as an active ingredient of an antimicrobial feed additive or feed composition, including any compound, preparation, mixture, or composition suitable for, or intended for intake by an animal such as a chicken, turkey, pig or swine, cow, sheep, horse, etc. In such embodiments, the halymorin variant exhibits antimicrobial activity against Gram negative bacteria. In some embodiments, the halymorin variant is added in an amount, for example, ranging from about 0.01 to about 10.0%; from about 0.05 to about 5.0%; or from about 0.1 to about 1.0% (% meaning gram additive per 100 grams of feed). All ranges include the endpoints and are combinable.

In some embodiments, the halymorin variant is used to preserve or hygienize antimicrobial feed because the halymorin variants exhibit antimicrobial activity against one or more species and/or strains of bacteria such as Salmonella enterica and Escherichia coli, which cause severe food-borne illnesses. The halymorin variant can be added directly to the animal feed in a treatment process of feed at levels ranging, for example, from 0.01 to 10.0%; or from 0.05 to 5.0%; or from 0.1 to 1.0%. In this context, the percent (%) refers to gram additive per 100 grams of a composition. All ranges include the endpoints and are combinable.

In various embodiments, the halymorin variant is used to prevent or treat one or more animals deemed to be at risk of bacterial infection or having already acquired (e.g., diagnosed with) a bacterial infection. In various embodiments, the halymorin variant has one or more advantages over existing antibiotics, including improved efficiency, ease of manufacture, lower material cost, and lower consumer price. In some embodiments, the halymorin variant comprises a sequence of 18 or 20 amino acids that can be formulated to be introduced into the body of an animal by itself, or as part of a polypeptide, or expressed from a DNA or RNA oligonucleotide. In some embodiments, the halymorin variant is effective at killing bacteria and has a higher killing efficiency than that of existing antibiotics, while also being safe to the animal that it may be administered to.

### EXAMPLES

### Strains, Reagents, Plasmids, Enzymes, and Growth Media

Chemicals were obtained from Sigma-Aldrich Co. (St. Louis, MO). Peptone and yeast extract, difc tryptic soy agar (TSA) and tryptic soy broth (TSB) (pancreatic digest of casein 15 g/L, papaic digest of soybean 5 g/L, sodium chloride 5 g/L, agar 15 g/L), LB broth (luria low salt), Nutrient broth (beef extract 3 g/L, peptone 5 g/L), and Brucella broth (tryptone 10 g/L, peptone 10 g/L, dextrose 1 g/L, yeast extract 2 g/L, sodium chloride 5.0 g/L, sodium metabisulfite. 0.1 g/L) were obtained from Thermo Fisher Scientific Inc. (Pittsburgh, PA). Restriction enzymes, Phusion high-fidelity DNA polymerase, and T4 ligase were obtained from New England Biolabs (Ipswich, NY). Escherichia coli DH5a, P. pastoris X33, and vectors pCR-blunt and pPicZalpha were obtained from Invitrogen (San Diego, CA). Minimal dextrose (MD) medium, minimal methanol (MM) medium, buffered glycerol complex (BMGY) medium, buffered methanol complex (BMMY) medium, and fermentation Basal Salts medium (BSM) were prepared according to the manual of Pichia Expression kit (Life Technologies Corp., USA).

The bacterial strains, including Campylobacter jejuni ATCC 12824, Campylobacter jejuni ATCC 33560, Escherichia coli ATCC 25922, Escherichia coli ATCC 33849, Escherichia coli ATCC 10798, Escherichia coli ATCC 700891, Salmonella enterica subsp. enterica serovar typhimurium ATCC 29630, Salmonella enterica subsp. enterica serovar choleraesuis ATCC 13312, Klebsiella pneumoniae ATCC 13883, and Enterobacter cloacae ATCC 13047, were obtained from ATCC (Manassas, VA).

### Construction of Expression Plasmids

The halymorin and halymorin variants were developed according to codon usage bias and guanine-cytosine (GC) content of P. pastoris using Genscript's OptimumGen designing tool (Piscataway, NJ). The designed halymorin and halymorin variants were synthesized by Eton Bioscience (Boston, MA) and subcloned into pPicZalpha vector by Life Technologies Corp. (USA). The resulting expression plasmid pPicZa was confirmed by restriction digestion and DNA sequencing (Eton Bioscience, USA).

### Yeast Transformation and Screening of Recombinant Pichia Strains

The plasmid pPicZa was linearized with Pme I restriction enzyme (Thermo Scientific) and then transformed into P. pastoris X33 by electroporation according to the manufacturer's instructions (Life Technologies Corp., USA). Transformants were screened on yeast extract peptone dextrose (YPD) (1% yeast extract, 2% peptone, 2% glucose) plates containing 100 μg/ml Zerocin (Life Technologies Corp., USA). The positive recombinants were analyzed by genomic polymerase chain reaction (PCR) with 5′ AOX and 3′ AOX primers. The recombinants identified by PCR were further screened in 125 mL shaken flasks. These strains were inoculated into 5 mL buffered glycerol complex medium (BMGY) (1% yeast extract, 2% peptone, 1.34% yeast nitrogen broth (YNB), 4×10⁻⁵% biotin, 1% glycerol and 100 mM potassium phosphate, pH 6.0) and cultured for 24 hours at 30° C. in 50 mL shaker flasks in a shaking incubator (250 rpm). After culture reaches an OD600=6, 1 mL of culture was transferred to a 125 mL shaker flask containing 10 mL buffered methanol-complex medium (BMMY) (1% yeast extract, 2% peptone, 1.34% YNB, 4×10⁻⁵% biotin, 0.5% methanol and 100 mM potassium phosphate, pH 6.0) and cultured for 24 hours at 30° C. (250 rpm). The enzyme expression was induced by adding 100% methanol to a final concentration of 0.5% methanol. The supernatant was collected by centrifugation at 12,000 rpm for 10 min (at 4° C.) for antimicrobial activity assay. The expressed peptides were analyzed by Tricine-SDS-PAGE.

### Purification of Expressed Peptides

The fermentation supernatant was precipitated with 40-45% ammonium sulfate. The precipitated peptides were centrifuged at 15000×g for 30 minutes. The pellets were resuspended with deionized water and purified using a Sephadex G-25 column and eluted with deionized water at a rate of 0.5 mL/min. The peak absorbance fractions were pooled for subsequent antimicrobial assays.

### Antimicrobial Activity Assay

The antimicrobial activity of purified halymorin and halymorin variants were analyzed using an inhibition zone assay. Test strains of E. coli ATCC 10798 were grown to OD600=0.5 at 37° C. in LB broth. A total of 100 μL of the cell suspension was inoculated into 20 mL of preheated LB agar medium (at about 42° C.) containing 1.5% agar. The medium was rapidly mixed and poured into the Petri dish (100 mm). Next, 5 mm holes were punched into the agar media plate with a glass capillary and 50 μL samples of a solution containing 10 μg of halymorin or a halymorin variant were dropped into the holes. Ampicillin (1 μg) was used as a positive control and sterile phosphate-buffered saline (PBS) was used a negative control. After incubation at 37° C. for 16-18 hours, the zones of growth inhibition were measured.

### The Minimal Inhibitory Concentration (MIC) Assay

Minimal inhibitory concentration assays (MIC, expressed as μg/mL) against different microorganisms were performed according to the protocol described in Clincial and Laboratory Standards Institute (CLSI) Methods for Dilution Antimicrobial Susceptibility Testing for Bacteria; Approved Standard-Eleventh Edition (2012). The tested bacteria: E. coli ATCC 25922, E. coli ATCC 33849, E. coli ATCC 10798, E. coli ATCC 700891, S. typhimurium ATCC 29630, S. choleraesuis ATCC 13312, K. pneumonica ATCC 13883 and E. cloacae ATCC 13047, were grown to OD600=0.5 at 37° C. in LB, TSB, or Nutrient broth. The bacterial cultures were diluted with medium to 10⁴-10⁶ CFU/mL. Then, a 10 μL peptide solution of various concentrations were added to 90 μL diluted culture fluid containing testing strains, resulting in a total volume of 100 μL. The 96-well microplates were incubated at 37° C. for 16 hours, and absorbance at 600 nm were taken to determine MIC. The MIC value was defined as the lowest peptide concentration that completely prevented growth using a microtiter optical plate reader.

The C. jejuni ATCC 12824 and C. jejuni ATCC 33560 strains were streaked on BB agar and grown microacrobically at 37° C. for 48 hours, then the cells were harvested in 2 mL BB media and diluted in the same medium to the appropriate concentration (OD600 nm=0.05). Then, 10 μL peptides with various concentrations was added to 90 μL diluted culture fluid containing the testing strains, resulting in a total volume of 100 μL. The 96-well microplates were incubated at 37° C. for 40 h, and absorbance at 600 nm were taken to determine MIC. The MIC value was defined as the lowest peptide concentration that completely prevented growth using a microtiter optical plate reader. All cultures were grown under microacrobic atmosphere (CampyGen, Fisher Scientific) at 37° C. with 150 rpm shaking.

### Hemolytic Assay

The hemolytic toxicity of halymorin and the halymorin variants Hal01, Hal05, and Hal10 were determined using 2% suspensions of human erythrocytes (Medix Biochemica, St. Louis, MO). Halymorin and the halymorin variants were diluted to concentrations of 250, 125, 62.5, 32, 16, 8, 4, 2, 1, and 0.5 μg/ml. The 100 μL of peptide solution and 100 μL of red blood cell suspension were mixed and added to the wells of a 96-well plate. PBS was used as a negative control and Triton X-100 was used as a positive control.

The samples were incubated at 37° C. for 60 minutes and gently stirred during the incubation period. The samples were then centrifuged at 2000 rpm for 5 minutes. A total of 100 μL of the supernatant in each well was transferred to a new 96-well plate and absorbance was measured at 490 nm using a microplate reader (Molecular Devices, USA).

### Example 1. Designed Halymorin Peptide Variants

Halymorin is an insect antimicrobial peptide consisting of 20 amino acids. The peptide has a molecular weight of 2256.6 Da and a +4.8 positive charge. The structure of halymorin has a β-hairpin Rana box, which is consistent with other β-hairpin antimicrobial peptides. To increase antimicrobial activity against Gram negative bacteria such as C. jejuni and E. coli, ten (10) halymorin peptide variants were designed and evaluated. The quality estimate ranges between 1 and 2 with higher values for better models.

The sequences of halymorin and the halymorin variants are shown in Table 1 along with their corresponding antimicrobial activities relative to the activity of halymorin. An activity of 1 corresponds to the activity of halymorin. An activity of 2 corresponds to an activity that is better than that of halymorin.

TABLE 1
Halymorin and halymorin peptide variants.

| SEQ | | | |
|---|---|---|---|
| ID | | | |
| NO: | Peptide | Sequence | Activity |
| 1 | halymorin | GSKPVPITYCNRKTGKCTRF | 1 |
| 2 | Hal01 | GSKPVPITYCNRQTGKCTRF | 2 |
| 3 | Hal02 | GSKPVPITYCNRNTGKCTRF | 1 |
| 4 | Hal03 | GSKPVPITYCNRQTGKCTRY | 2 |
| 5 | Hal04 | GSKPVPITYCNRQTGKCSRF | 2 |
| 6 | Hal05 | GKVPITYCNRQTGKCTRF | 2 |
| 7 | Hal06 | GSKPVPITYCNRNTGKCQRM | 1 |
| 8 | Hal07 | GKKKVPIIYCNRKTGVCKRM | 2 |
| 9 | Hal08 | GKVPIIYCNRNTGVCKRM | 2 |
| 10 | Hal09 | GSKKPVPIIYCNRQTGKCQRM | 2 |
| 11 | Hal10 | GSKKPVPIIYCNRNTGKCQRM | 2 |
| 12 | Hal11 | GSKKPVPIIYCNRLTGKCQRM | 1 |

### Example 2. Evaluation of Antimicrobial Activity

The halymorin and halymorin variants were expressed in P. pastoris X33. The supernatant was collected by centrifugation at 12,000 rpm for 10 minutes at 4° C. The fermentation supernatant was precipitated with 40-45% ammonium sulfate. The precipitated peptides were centrifuged at 15,000×g for 30 minutes. The pellets were resuspended with deionized water and purified with Sephadex G-25 columns.

The antimicrobial activity of purified halymorin and halymorin variants were analyzed using the inhibition zone assay. Test strains of E. coli ATCC 10798 were grown to OD600=0.5 at 37° C. in LB broth. A total of 100 μL of the cell suspension was inoculated into 20 mL of preheated LB broth agar medium (at about 42° C.) containing 1.5% agar. The medium was rapidly mixed and poured into the Petri dish (100 mm). The 5 mm holes were punched with a glass capillary and then filled with a 50 μL solution containing 10 μg of halymorin or halymorin variants. Ampicillin (1 μg) was used as a positive control and sterile PBS was used a negative control. After incubation at 37° C. for 16-18 hours, the zones of growth inhibition were measured.

### Example 3. Measuring Minimal Inhibitory Concentration

The halymorin variants according to SEQ ID NOs: 02, 06, 09, 10 and 12 exhibited particularly good antimicrobial activity and were selected for further analysis. The halymorin variants were tested using a minimal inhibitory concentration assay (MIC, expressed as μl/mL) following the protocol described in the Clincial and Laboratory Standards Institute (CLSI) Methods for Dilution Antimicrobial Susceptibility Testing for Bacteria; Approved Standard—Eleventh Edition (2012) against the following microorganisms: C. jejuni ATCC 33560; C. jejuni ATCC 12824; E. coli ATCC 33849; E. coli ATCC 25922; E. coli ATCC 10798; E. coli ATCC 700891; K. pneumonica ATCC 13883; S. typhimusium ATCC 29630; S. choleraesuis ATCC 13312; and E. cloacae ATCC 13047. The results are provided in Table 2.

Compared to the wildtype halymorin (SEQ ID NO: 1), the halymorin variants Hal05 (SEQ ID NO: 6), Hal08 (SEQ ID NO: 9), and Hal09 (SEQ ID NO: 10) exhibited improved antimicrobial activity (i.e., lower MIC values) against the bacteria C. jejuni ATCC 12824 and C. jejuni ATCC 33560. Compared to the wildtype halymorin (SEQ ID NO: 1), the halymorin variants Hal09 (SEQ ID NO: 10) and Hal10 (SEQ ID NO: 11) exhibited improved antimicrobial activity (i.e., lower MIC values) against E. coli, S. typhimurium, and S. choleraesuis. The Minimal inhibitory concentration (MIC) for halymorin and the halymorin variants against Gram-negative bacteria is shown in Table 2.

TABLE 2
MIC of halymorin and variants against Gram negative bacteria.
Minimal inhibitory concentration (MIC) (μg/ml)

| | SEQ ID NO | | | | | |
|---|---|---|---|---|---|---|
| Microbe | 1 | 2 | 6 | 9 | 10 | 11 |
| C. jejuni ATCC 33560 | 37.5 | 37.5 | 25.0 | 12.5 | 6.25 | 3.13 |
| C. jejuni ATCC 12824 | 37.5 | 37.5 | 25.0 | 12.5 | 6.25 | 3.13 |
| E. coli ATCC 33849 | 3.44 | 1.75 | 2.56 | 1.33 | 1.38 | 1.43 |
| E. coli ATCC 25922 | 4.68 | 1.75 | 3.80 | 2.56 | 1.38 | 0.71 |
| E. coli ATCC 10798 | 3.44 | 0.88 | 2.56 | 2.56 | 1.38 | 0.10 |
| E. coli ATCC 700891 | 4.28 | 1.75 | 2.56 | 3.33 | 0.18 | 0.18 |
| K. pneumonica ATCC 13883 | 2.14 | 0.88 | 1.25 | 0.33 | 0.10 | 0.10 |
| S. typhimusium ATCC 29630 | 1.57 | 0.44 | 0.63 | 2.56 | 0.69 | 0.10 |
| S. choleraesuis ATCC 13312 | 2.14 | 0.44 | 0.63 | 0.67 | 0.10 | 0.10 |
| E. cloacae ATCC 13047 | 9.15 | 3.50 | 5.40 | 2.56 | 0.35 | 0.36 |

### Example 4. Hemolytic Activity of Halymorin Peptides

For hemolysis assays, human erythrocytes were obtained from healthy donors, washed 3 times using sterilized PBS, and resuspended to a concentration of 2% (v/v) with PBS. The halymorin and halymorin variants Hal01, Hal05, and Hal10 were diluted to concentrations of 250, 125, 62.5, 32, 16, 8, 4, 2, 1, and 0.5 μg/ml. A 100 μL portion of each peptide solution was mixed with a 100 μL red blood cell suspension (Medix Biochemica, St Louis, USA) and then added to the wells of a 96-well plate. PBS was used as negative control and Triton X-100 was used as a positive control. The samples were incubated at 37° C. for 60 minutes and gently stirred during the incubation period. Then, the samples were centrifuged at 2000 rpm for 5 minutes. A total of 100 μL of the supernatant in each well was transferred to a new 96-well plate and absorbance was measured at 490 nm using a microplate reader (Molecular Devices, USA).

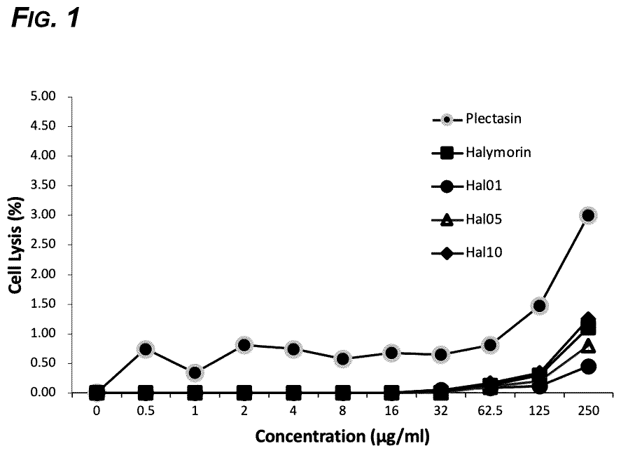

As shown in FIG. 1, halymorin and the halymorin variants Hal01, Hal05, and Hal10 exhibited no significant lysis of the human cells at any concentration of peptides tested (0.5-250 μg/ml). By contrast, plectasin exhibited lysis at nearly all concentrations, and the cell lysis increased significantly as the concentration was increased from 62.5 μg/ml to 250 μg/ml.

As described above, the antimicrobial peptide halymorin variants exhibit remarkable antibacterial effects against Gram negative bacteria without harmful side effects to human cells. The halymorin variants are effective active ingredients for feed additives, food preservatives, cosmetics, and/or pharmaceutical compositions.

A recitation of a range of values herein is merely intended to serve as a shorthand method of referring individually to each value falling within the range, unless otherwise indicated herein, and each separate value is incorporated into the specification as if it were individually recited herein. Thus, for example, a component provided in 1-3 grams refers to the component being provided in 1, 2, or 3 grams. As will be understood by one skilled in the art, ranges disclosed herein encompass any and all possible subranges and combinations of subranges thereof. Any listed range can be easily recognized as sufficiently describing the same range being broken down into at least equal halves, thirds, quarters, fifths, tenths, etc. As a non-limiting example, each range discussed herein can be readily broken down into a lower third, middle third and upper third, etc. As will also be understood by one skilled in the art, language such as “up to,” “at least,” “greater than,” “less than,” and the like include the number recited and refer to ranges which can be subsequently broken down into subranges as discussed above.

As used herein, the use of examples, or exemplary language (e.g., “such as”), is intended to illuminate the embodiments and does not pose a limitation on the scope of the claims unless otherwise stated. No language in the specification should be construed as indicating any non-claimed element as essential.

As used herein, the terms “about” and “substantially” will be understood by persons of ordinary skill in the art and will vary to some extent depending upon the context in which it is used. If there are uses of the term which are not clear to persons of ordinary skill in the art, given the context in which it is used, “about” and “substantially” will mean up to plus or minus 10% of the particular term.

Exemplary embodiments of the methods are described above in detail. The methods are not limited to the specific embodiments described herein, but rather, steps of the method may be utilized independently and separately from other steps described herein. All methods described herein can be performed in any suitable order unless otherwise indicated herein or otherwise clearly contradicted by context.

The foregoing embodiments are provided to aid in the understanding of the present disclosure, the true scope of which is set forth in the appended claims. This written description uses examples to disclose the present embodiments, including the best mode, and also to enable any person skilled in the art to practice the present embodiments, including performing any methods. The patentable scope of the present embodiments is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

I claim:

1. An antimicrobial peptide consisting of an amino acid sequence selected from the group consisting of SEQ ID NOs: 2-12.

2. The antimicrobial peptide according to claim 1, wherein the antimicrobial peptide has antimicrobial activity against Gram-negative bacteria.

3. The antimicrobial peptide according to claim 2, wherein the Gram-negative bacteria is Campylobacter jejuni.

4. The antimicrobial peptide according to claim 2, wherein the Gram-negative bacteria is Escherichia coli.

5. The antimicrobial peptide according to claim 2, wherein the Gram-negative bacteria is Salmonella enterica subsp. enterica serovar typhimurium.

6. The antimicrobial peptide according to claim 2, wherein the Gram-negative bacteria is Salmonella enterica subsp. enterica serovar choleraesuis.

7. The antimicrobial peptide according to claim 2, wherein the Gram-negative bacteria is Klebsiella pneumonica.

8. The antimicrobial peptide according to claim 2, wherein the Gram-negative bacteria is Enterobacter cloacae.

9. An antimicrobial peptide composition comprising the antimicrobial peptide of claim 1 as an active ingredient.

10. A pharmaceutical composition comprising the antimicrobial peptide of claim 1 as an active ingredient.

11. An antimicrobial food additive comprising the antimicrobial peptide of claim 1 as an active ingredient.

12. A cosmetic composition comprising the antimicrobial peptide of claim 1 as an active ingredient.

13. A hygiene product comprising the antimicrobial peptide of claim 1 as an active ingredient.

14. A method of treating an animal having an infectious disease caused by bacteria, the method comprising administering a pharmaceutical composition comprising the antimicrobial peptide of claim 1 as an active ingredient.

15. The method according to claim 14, wherein the antimicrobial peptide is the amino acid sequence of SEQ ID NO: 2.

16. The method according to claim 14, wherein the antimicrobial peptide is the amino acid sequence of SEQ ID NO: 3.

17. The method according to claim 14, wherein the antimicrobial peptide is the amino acid sequence of SEQ ID NO: 6.

18. The method according to claim 14, wherein the antimicrobial peptide is the amino acid sequence of SEQ ID NO: 9.

19. The method according to claim 14, wherein the antimicrobial peptide is the amino acid sequence of SEQ ID NO: 10.

20. The method according to claim 14, wherein the antimicrobial peptide is the amino acid sequence of SEQ ID NO: 11.


SEQUENCE LISTING (12 sequences)

SEQ ID NO: 1 - protein, 20 aa, Halyomorpha sp.
GSKPVPITYCNRKTGKCTRF

SEQ ID NO: 2 - protein, 20 aa, synthetic construct
  REGION 1..20: note = synthetic halymorin variant
GSKPVPITYCNRQTGKCTRF

SEQ ID NO: 3 - protein, 20 aa, synthetic construct
  REGION 1..20: note = synthetic halymorin variant
GSKPVPITYCNRNTGKCTRF

SEQ ID NO: 4 - protein, 20 aa, synthetic construct
  REGION 1..20: note = synthetic halymorin variant
GSKPVPITYCNRQTGKCTRY

SEQ ID NO: 5 - protein, 20 aa, synthetic construct
  REGION 1..20: note = synthetic halymorin variant
GSKPVPITYCNRQTGKCSRF

SEQ ID NO: 6 - protein, 18 aa, synthetic construct
  REGION 1..18: note = synthetic halymorin variant
GKVPITYCNRQTGKCTRF

SEQ ID NO: 7 - protein, 20 aa, synthetic construct
  REGION 1..20: note = synthetic halymorin variant
GSKPVPITYCNRNTGKCQRM

SEQ ID NO: 8 - protein, 20 aa, synthetic construct
  REGION 1..20: note = synthetic halymorin variant
GKKKVPIIYCNRKTGVCKRM

SEQ ID NO: 9 - protein, 18 aa, synthetic construct
  REGION 1..18: note = synthetic halymorin variant
GKVPIIYCNRNTGVCKRM

SEQ ID NO: 10 - protein, 21 aa, synthetic construct
  REGION 1..21: note = synthetic halymorin variant
GSKKPVPIIYCNRQTGKCQRM

SEQ ID NO: 11 - protein, 21 aa, synthetic construct
  REGION 1..21: note = synthetic halymorin variant
GSKKPVPIIYCNRNTGKCQRM

SEQ ID NO: 12 - protein, 21 aa, synthetic construct
  REGION 1..21: note = synthetic halymorin variant
GSKKPVPIIYCNRLTGKCQRM